(19) United States
(12) Patent Application Publication
(10) Pub. No.: US 2025/0107947 A1
(43) Pub. Date: Apr. 3, 2025

(54) Adult Diaper Device

(71) Applicant: Patricia Sharlene Miller, Ragley, LA (US)

(72) Inventor: Patricia Sharlene Miller, Ragley, LA (US)

(21) Appl. No.: 18/887,060

(22) Filed: Sep. 17, 2024

Related U.S. Application Data
(60) Provisional application No. 63/587,234, filed on Oct. 2, 2023.

(51) Int. Cl. A61F 13/49; A61F 13/496

(52) U.S. Cl. CPC A61F 13/49011; A61F 13/4963

(57) ABSTRACT

An adult diaper device is disclosed which is a specialized adult diaper to maintain comfort and prevent leaks. The adult diaper device comprises a body component that is configured as a pair of underwear with extended legs. The extended leg portions are absorbent, nonrestrictive, and elastic for comfort. The body component comprises absorbent material in the crotch, back, and inside leg portions, such that the absorbent material extends down the legs, toward the knee, and goes up the back toward a back of a user to maximize coverage and help prevent any leaks and embarrassing stains on the clothing. Further, the body component comprises an elastic waistband that is non-restrictive and helps maintain comfort while wearing the device throughout the day. The body component also comprises a tear-away seam at the sides for easy on and off of the device.

### CROSS-REFERENCE TO RELATED APPLICATION

The present application claims priority to, and the benefit of, U.S. Provisional Application No. 63/587,234, which was filed on Oct. 2, 2023, and is incorporated herein by reference in its entirety.

### FIELD OF THE INVENTION

The present invention relates generally to the field of adult diaper devices. More specifically, the present invention relates to a specialized adult diaper designed to extend down toward the knees to maximize coverage and absorbency for accidental leaks. Accordingly, the present disclosure makes specific reference thereto. Nonetheless, it is to be appreciated that aspects of the present invention are also equally applicable to other like applications, devices, and methods of manufacture.

### BACKGROUND

By way of background, this invention relates to improvements in adult diaper devices. Generally, people who suffer from IBS, IBS-C, IBS-D and other incontinence issues may need to wear adult diapers throughout the day to help prevent embarrassing stains and leaks. However, despite functioning to absorb solid and liquid waste, standard adult diapers may not offer enough coverage to fully prevent a leak. Further, any liquid or solid waste leaks can be incredibly embarrassing, leaving people with very little self-confidence. Additionally, absorbency in the rear of the diaper may be lacking, and fit at the hips may be inadequate.

In addition, various types of diapers have been used for adults, especially those persons who are incontinent due to mental or emotional problems, as well as physical illnesses including bladder or urinary disorders, prostate involvement, or senility. Persons having these problems can have useful lives if embarrassment is avoided by employing an adult diaper capable of being comfortably employed with conventional clothing. However, these diapers do not fully prevent leaks and tend to embarrass users.

Accordingly, there is a demand for an improved adult diaper device that provides maximum coverage and absorbency for accidental leaks. More particularly, there is a demand for an adult diaper device that improves self-confidence and self-worth for those with incontinence issues.

Therefore, there exists a long-felt need in the art for an adult diaper device that provides users with a specialized adult diaper designed to extend down toward the knees to maximize coverage and absorbency for accidental leaks. There is also a long-felt need in the art for an adult diaper device that features absorbent layers on the crotch, sides, front, back, and inside of the leg to reduce the chance of a leak by absorbing all bodily fluids. Further, there is a long-felt need in the art for an adult diaper device that includes a comfortable, adjustable elastic band at the leg. Moreover, there is a long-felt need in the art for a device that also includes a tear-away seam at the sides to maintain comfort and offer easy on and off for the diaper. Further, there is a long-felt need in the art for an adult diaper device that improves confidence and self-worth for those who suffer from severe incontinence issues. Finally, there is a long-felt need in the art for an adult diaper device that prevents embarrassing stains on clothing.

The subject matter disclosed and claimed herein, in one embodiment thereof, comprises an adult diaper device. The device is a specialized adult diaper to maintain comfort and prevent leaks. The adult diaper device comprises a body component that is configured as a pair of underwear with extended legs. The extended leg portions are absorbent, nonrestrictive, and elastic for comfort. The body component comprises absorbent material in the crotch, back, and inside leg portions, such that the absorbent material extends down the legs, toward the knee, and goes up the back toward a back of a user to maximize coverage and help prevent any leaks and embarrassing stains on the clothing. Further, the body component comprises an elastic waistband that is non-restrictive and helps maintain comfort while wearing the device throughout the day. The body component also comprises a tear-away seam at the sides for easy on and off of the device.

In this manner, the adult diaper device of the present invention accomplishes all of the foregoing objectives and provides users with a comfortable adult diaper for maximum coverage. The device is an adult diaper that has extended leg portions. The device can be easily put on and removed, as needed.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an adult diaper device. The device is a specialized adult diaper to maintain comfort and prevent leaks. The adult diaper device comprises a body component that is configured as a pair of underwear with extended legs. The body component comprises absorbent material in the crotch, back, and inside leg portions, such that the absorbent material extends down the legs, toward the knee, and goes up the back toward a back of a user to maximize coverage and help prevent any leaks and embarrassing stains on the clothing.

In one embodiment, the adult diaper device is a specialized garment that is designed to be worn as a conventional pair of underwear, with leg portions that extend down toward the user's knees to maximize coverage and absorbency for accidental leaks due to incontinence, IBS, or other typical issues. Further, the adult diaper device relates to a disposable underwear garment for men and women with IBS and other incontinence issues, to improve the comfort and absorption capacity of disposable underwear and provide additional absorbency where some is lacking.

In one embodiment, the adult diaper device comprises a body component configured as a conventional pair of underwear, with a central portion and two leg portions. The central portion has an upper region and a lower region that covers a user's waist and crotch area respectively. The lower region of the central portion defines a pair of upper thigh openings. The upper region of the central portion defines a waist opening. Specifically, a waistband extends from the upper region of the central portion, to secure the device around a user's waist.

The two leg portions downwardly depend from the pair of upper thigh openings disposed at the lower region of the central portion. The two leg portions have a top end and a bottom end. The top end of the leg sleeve is attached to the central portion by elastic material, seams, sewing directly, etc. The bottom end defines leg openings that terminate on the thigh of the user's leg, preferably at about the mid to lower thigh. Further, the edges of the central portion and leg portions may be seam-attached about their outer edges. Various attachment means, such as cotton thread, polyester thread, monofilament, and the like may be used in seam-attaching the outer edges of the body component.

In one embodiment, the waistband of the body component is an elastic waistband that conforms to a user's waist. Specifically, a continuous, or uninterrupted, elastic waistband may be employed to secure the body component using such means as an elastic band, a drawstring, or equivalent means. The elastic waistband is non-restrictive and helps maintain comfort while wearing the device throughout the day. The waistband also forms a tight seal around a user's waist to prevent leaks. Specifically, elastic material is incorporated into the waistband, which encircles the waist of the user. The elastic waistband forms a seal to the user's upper torso to prevent waste matter from seeping up and out the waist opening, during use.

In one embodiment, the sides of the body component comprise a tear-away seam for fast and easy removal. The tear-away seam can be comprised of tear-away fasteners, hook and loop fasteners, snaps, etc., or any other suitable fasteners as is known in the art. The tear-away seam can be re-closable or permanently removed. The tear-away seams would run the entire length of one side of the body component. In another embodiment, the tear-away seams run the entire length of both sides of the body component. The tear-away seams at the sides help maintain comfort of the device and offer easy on and off for the device.

Accordingly, the sides of the body component may be separated at the waistband and may be releasably joined to form a functioning disposable body component by side-mounted fastening means (i.e., hook and loop fastener, button, or snap closure means, etc.). Further, multiple fastening means may be used to reinforce the integrity of the body component or to enhance the aesthetic qualities of the adult diaper device. Accordingly, a user may adjust the fastening means to a desired configuration to maximize convenience and comfort when donning the adult diaper device. The single-side closure and double-side closure configurations of the adult diaper device allow a user to remove and/or replace the adult diaper device without having to completely remove a lower garment such as pants or shorts beforehand.

In one embodiment, the body component utilizes elastic material or any other resilient material to provide tight seals and conform to the user's body. For example, the elastic material, disposed between the two leg portions and the central portion, forms a tight seal on the upper thigh of the user. Likewise, the bottom ends of the two leg portions utilize elastic material to form a tight fit on the user's mid to lower thigh. Thus, waste matter excreted by the user is retained within the body component due to the seal formed at the user's upper thighs by the elastic material, as well as the elastic waistband. If, however, waste matter leaks past the elastic material, then the seal provided at the bottom end of the leg sleeves would prevent waste matter from leaking out the leg openings. Here, the elastic material and the elastic incorporated at the bottom end of the leg sleeves provide double protection from waste matter leaking out the leg openings.

In one embodiment, the body component has an exterior surface and an interior surface. The interior surface is dimensioned and configured to contact the user's body, crotch, and legs while the exterior surface is designed to contact clothing worn by the user. The exterior surface comprises non-absorbent material and is impermeable to liquids. The exterior surface is typically comprised of a fluid impervious waterproof plastic material, such as polyethylene or polypropylene, or non-woven hydrophobic fibers of polyethylene or polypropylene or a combination thereof. Whereas, the interior surface of the body component comprises absorbent materials. The absorbent materials give the device a maximum thickness of approximately about ½ to ¾ inch. The absorbent materials typically include a matrix of superabsorbent polymers and wood fluff pulp. However, upon reading this specification, it should be appreciated that, under appropriate circumstances, considering such issues as user preferences, design preference, structural requirements, marketing preferences, cost, available materials, technological advances, etc., absorbent material arrangements such as, for example, cotton, hemp, wool, bleached wood pulp, man-made absorbent materials, etc., may be sufficient.

In one embodiment, typically, absorbent materials are positioned in the crotch area, and up the back of the lower region, as well as within the two leg portions. Thus, the absorbent materials extend up towards a user's back and down the user's legs (toward the knees), to maximize coverage and help prevent any leaks and embarrassing stains on clothing.

In one embodiment, the adult diaper device is effectively a single-use article that may be discarded after one absorbent use. Preferably, the adult diaper device may be manufactured in sizes similar to those currently available in brands of traditional multi-use underwear for men and/or women. Accordingly, the adult diaper device may be manufactured and provided for sale in a wide variety of sizes and shapes for a wide assortment of applications and leak protections.

In yet another embodiment, the adult diaper device comprises a plurality of indicia.

In yet another embodiment, a method of preventing embarrassing accidents and leaks when suffering with incontinence issues and IBS is disclosed. The method includes the steps of providing an adult diaper device comprising a body component with extended leg portions. The method also comprises inserting a user's legs into the extended leg portions and pulling up the device. Further, the method comprises wearing the device throughout the day. The method also comprises absorbing any leaks or accidents. Finally, the method comprises utilizing the tear-away seam to remove the device after leaks or accidents for disposing in the trash.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front perspective view of one embodiment of the adult diaper device of the present invention showing the device with extended leg portions in accordance with the disclosed architecture;

FIG. 2 illustrates a side perspective view of one embodiment of the adult diaper device of the present invention showing the elastic waistband of the device in accordance with the disclosed architecture;

FIG. 3 illustrates a front perspective view of one embodiment of the adult diaper device of the present invention showing the absorbency of the device in accordance with the disclosed architecture;

FIG. 4 illustrates a side perspective view of one embodiment of the adult diaper device of the present invention showing the tear-away seam in accordance with the disclosed architecture;

FIG. 5 illustrates a front perspective view of one embodiment of the adult diaper device of the present invention in use in accordance with the disclosed architecture; and

FIG. 6 illustrates a flowchart showing the method of preventing embarrassing accidents and leaks when suffering with incontinence issues and IBS in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for an adult diaper device that provides users with a specialized adult diaper designed to extend down toward the knees to maximize coverage and absorbency for accidental leaks. There is also a long-felt need in the art for an adult diaper device that features absorbent layers on the crotch, sides, front, back, and inside of the leg to reduce the chance of a leak by absorbing all bodily fluids. Further, there is a long-felt need in the art for an adult diaper device that includes a comfortable, adjustable elastic band at the leg. Moreover, there is a long-felt need in the art for a device that also includes a tear-away seam at the sides to maintain comfort and offer easy on and off for the diaper. Further, there is a long-felt need in the art for an adult diaper device that improves confidence and self-worth for those who suffer from severe incontinence issues. Finally, there is a long-felt need in the art for an adult diaper device that prevents embarrassing stains on clothing.

The present invention, in one exemplary embodiment, is a novel adult diaper device. The adult diaper device comprises a body component that is configured as a pair of underwear with extended legs. The extended leg portions are absorbent, nonrestrictive, and elastic for comfort. The body component comprises absorbent material in the crotch, back, and inside leg portions, such that the absorbent material extends down the legs, toward the knee, and goes up the back toward a back of a user to maximize coverage and help prevent any leaks and embarrassing stains on the clothing. Further, the body component comprises an elastic waistband that is non-restrictive and helps maintain comfort while wearing the device throughout the day. The body component also comprises a tear-away seam at the sides for easy on and off of the device. The present invention also includes a novel method of preventing embarrassing accidents and leaks when suffering with incontinence issues and IBS. The method includes the steps of providing an adult diaper device comprising a body component with extended leg portions. The method also comprises inserting a user's legs into the extended leg portions and pulling up the device. Further, the method comprises wearing the device throughout the day. The method also comprises absorbing any leaks or accidents. Finally, the method comprises utilizing the tear-away seam to remove the device after leaks or accidents for disposing in the trash.

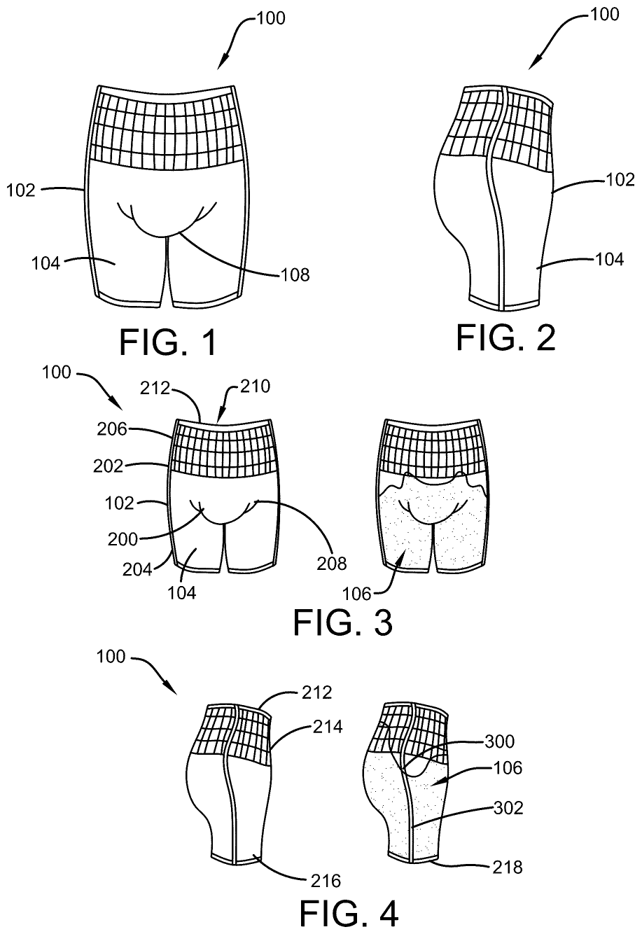

Referring initially to the drawings, FIGS. 1-2 illustrate a perspective view of one embodiment of the adult diaper device 100 of the present invention. In the present embodiment, the adult diaper device 100 is an improved adult diaper device 100 that provides a user 114 with a specialized adult diaper designed to maintain comfort and prevent leaks and accidents. Specifically, the adult diaper device 100 comprises a body component 102 that is configured as a pair of underwear with extended leg portions 104. The body component 102 comprises absorbent material 106 in the crotch area 108, back 110, and inside leg portions 104, such that the absorbent material 106 extends down the legs 104, toward the knee 112, and goes up the back 110 toward a back of a user 114 to maximize coverage and help prevent any leaks and embarrassing stains on the clothing 116.

Generally, the adult diaper device 100 is a specialized garment that is designed to be worn as a conventional pair of underwear, with leg portions 104 that extend down toward the user's knees 112 to maximize coverage and absorbency for accidental leaks due to incontinence, IBS, or other typical issues. Further, the adult diaper device 100 relates to a disposable underwear garment for men and women with IBS and other incontinence issues, to improve the comfort and absorption capacity of disposable underwear and provide additional absorbency where some is lacking.

Further, the adult diaper device 100 comprises a body component 102 configured as a conventional pair of underwear, with a central portion 200 and two leg portions 104. The central portion 200 has an upper region 202 and a lower region 204 that covers a user's waist 206 and crotch area 108 respectively. The lower region 204 of the central portion 200 defines a pair of upper thigh openings 208. The upper region 202 of the central portion 200 defines a waist opening 210. Specifically, a waistband 212 extends from the upper region 202 of the central portion 200, to secure the device 100 around a user's waist 206.

Additionally, the two leg portions 104 downwardly depend from the pair of upper thigh openings 208 disposed at the lower region 204 of the central portion 200. The two leg portions 104 have a top end 214 and a bottom end 216. The top end 214 of the leg sleeve 104 is attached to the central portion 200 by elastic material, seams 218, sewing directly, etc. The bottom end 216 defines leg openings 104 that terminate on the thigh of the user's leg 220, preferably at about the mid to lower thigh 222. Further, the edges 224 of the central portion 200 and leg portions 104 may be seam-attached 218 about their outer edges 224. Various attachment means, such as cotton thread, polyester thread, monofilament, and the like may be used in seam-attaching the outer edges 224 of the body component 102.

As shown in FIGS. 3-4, the waistband 212 of the body component 102 is an elastic waistband 212 that conforms to a user's waist 206. Specifically, a continuous, or uninterrupted, elastic waistband 212 may be employed to secure the body component 102 using such means as an elastic band, a drawstring, or equivalent means. The elastic waistband 212 is non-restrictive and helps maintain comfort while wearing the device 100 throughout the day. The waistband 212 also forms a tight seal around a user's waist 206 to prevent leaks. Specifically, elastic material is incorporated into the waistband 212, which encircles the waist 206 of the user 114. The elastic waistband 212 forms a seal to the user's upper torso to prevent waste matter from seeping up and out the waist opening 210, during use.

Further, the sides 300 of the body component 102 comprise a tear-away seam 302 for fast and easy removal. The tear-away seam 302 can be comprised of tear-away fasteners, hook and loop fasteners, snaps, etc., or any other suitable fasteners as is known in the art. The tear-away seam 302 can be re-closable or permanently removed. The tear-away seams 302 would run the entire length of one side 300 of the body component 102. In another embodiment, the tear-away seams 302 run the entire length of both sides 300 of the body component 102. The tear-away seams 302 at the sides 300 help maintain comfort of the device 100 and offer easy on and off for the device 100.

Accordingly, the sides 300 of the body component 102 may be separated at the waistband 212 and may be releasably joined to form a functioning disposable body component 102 by side-mounted fastening means (i.e., hook and loop fastener, button, or snap closure means, etc.). Further, multiple fastening means may be used to reinforce the integrity of the body component 102 or to enhance the aesthetic qualities of the adult diaper device 100. Accordingly, a user 114 may adjust the fastening means to a desired configuration to maximize convenience and comfort when donning the adult diaper device 100. The single-side closure and double-side closure configurations of the adult diaper device 100 allow a user 114 to remove and/or replace the adult diaper device 100 without having to completely remove a lower garment such as pants or shorts beforehand.

Furthermore, the body component 102 utilizes elastic material or any other resilient material to provide tight seals and conform to the user's body. For example, the elastic material, disposed between the two leg portions 104 and the central portion 200, forms a tight seal on the upper thigh 222 of the user 114. Likewise, the bottom ends 216 of the two leg portions 104 utilize elastic material to form a tight fit on the user's mid to lower thigh 222. Thus, waste matter excreted by the user 114 is retained within the body component 102 due to the seal formed at the user's upper thighs 222 by the elastic material, as well as the elastic waistband 212. If, however, waste matter leaks past the elastic material, then the seal provided at the bottom end 216 of the leg sleeves 104 would prevent waste matter from leaking out the leg openings 104. Here, the elastic material and the elastic incorporated at the bottom end 216 of the leg sleeves 104 provide double protection from waste matter leaking out the leg openings 104.

Additionally, the body component 102 has an exterior surface 304 and an interior surface 306. The interior surface 306 is dimensioned and configured to contact the user's body, crotch area 108, and legs 220 while the exterior surface 304 is designed to contact clothing worn by the user 114. The exterior surface 304 comprises non-absorbent material and is impermeable to liquids. The exterior surface 304 is typically comprised of a fluid impervious waterproof plastic material, such as polyethylene or polypropylene, or non-woven hydrophobic fibers of polyethylene or polypropylene or a combination thereof. Whereas, the interior surface 306 of the body component 102 comprises absorbent materials 106. The absorbent materials 106 give the device 100 a maximum thickness of approximately about ½ to ¾ inch. The absorbent materials 106 typically include a matrix of superabsorbent polymers and wood fluff pulp. However, upon reading this specification, it should be appreciated that, under appropriate circumstances, considering such issues as user preferences, design preference, structural requirements, marketing preferences, cost, available materials, technological advances, etc., absorbent material arrangements such as, for example, cotton, hemp, wool, bleached wood pulp, man-made absorbent materials, etc., may be sufficient.

Typically, absorbent materials 106 are positioned in the crotch area 108, and up the back 110 of the lower region 204, as well as within the two leg portions 104. Thus, the absorbent materials 106 extend up towards a user's back 110 and down the user's legs 220 (toward the knees 112), to maximize coverage and help prevent any leaks and embarrassing stains on clothing.

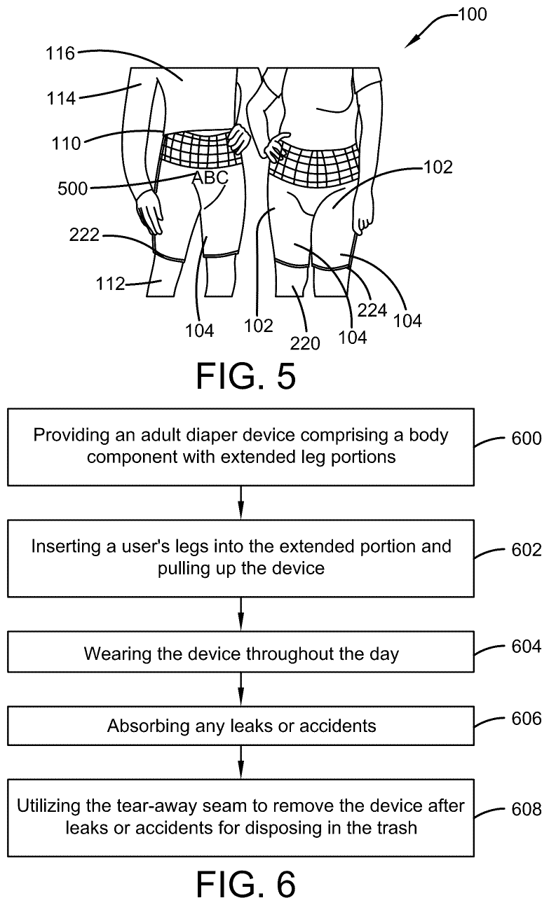

As shown in FIG. 5, the adult diaper device 100 is effectively a single-use article that may be discarded after one absorbent use. Preferably, the adult diaper device 100 may be manufactured in sizes similar to those currently available in brands of traditional multi-use underwear for men and/or women. Accordingly, the adult diaper device 100 may be manufactured and provided for sale in a wide variety of sizes and shapes for a wide assortment of applications and leak protections.

In yet another embodiment, the adult diaper device 100 comprises a plurality of indicia 500. The body component 102 of the device 100 may include advertising, a trademark, or other letters, designs, or characters, printed, painted, stamped, or integrated into the body component 102, or any other indicia 500 as is known in the art. Specifically, any suitable indicia 500 as is known in the art can be included, such as, but not limited to, patterns, logos, emblems, images, symbols, designs, letters, words, characters, animals, advertisements, brands, etc., that may or may not be underwear, adult diaper, or brand related.

FIG. 6 illustrates a flowchart of the method of preventing embarrassing accidents and leaks when suffering with incontinence issues and IBS. The method includes the steps of at 600, providing an adult diaper device comprising a body component with extended leg portions. The method also comprises at 602, inserting a user's legs into the extended leg portions and pulling up the device. Further, the method comprises at 604, wearing the device throughout the day. The method also comprises at 606, absorbing any leaks or accidents. Finally, the method comprises at 608, utilizing the tear-away seam to remove the device after leaks or accidents for disposing in the trash.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “adult diaper device”, “diaper device”, and “device” are interchangeable and refer to the adult diaper device 100 of the present invention.

Notwithstanding the foregoing, the adult diaper device 100 of the present invention can be of any suitable size and configuration as is known in the art without affecting the overall concept of the invention, provided that it accomplishes the above-stated objectives. One of ordinary skill in the art will appreciate that the adult diaper device 100 as shown in FIGS. 1-6 is for illustrative purposes only, and that many other sizes and shapes of the adult diaper device 100 are well within the scope of the present disclosure. Although the dimensions of the adult diaper device 100 are important design parameters for user convenience, the adult diaper device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An adult diaper device that maintains comfort and prevents leaks and accidents, the adult diaper device comprising:
a body component;
a pair of leg portions; and
absorbent material;
wherein the body component is configured as a pair of underwear with the pair of leg portions attached, which extend down a user's legs during wear; and
further wherein the body component comprises absorbent material in a crotch area, back, and inside the pair of leg portions to prevent leaks and maximize coverage.

2. The adult diaper device of claim 1, wherein the body component comprises a central portion with an upper region that extends up to cover a user's waist and a lower region that covers a user's crotch area.

3. The adult diaper device of claim 2, wherein the lower region of the central portion defines a pair of upper thigh openings.

4. The adult diaper device of claim 3, wherein the pair of leg portions downwardly depend from the pair of upper thigh openings disposed at the lower region of the central portion.

5. The adult diaper device of claim 4, wherein the pair of leg portions have a top end and a bottom end, with the top end attached to the central portion and the bottom end defines leg openings that terminate on a thigh of a user's leg.

6. The adult diaper device of claim 5, wherein the body component comprises an elastic waistband that conforms to a user's waist.

7. The adult diaper device of claim 6, wherein one side of the body component comprises a tear-away seam for fast and easy removal.

8. The adult diaper device of claim 7, wherein both sides of the body component comprise a tear-away seam.

9. The adult diaper device of claim 8, wherein the body component comprises an exterior surface and an interior surface.

10. The adult diaper device of claim 9, wherein the interior surface is dimensioned and configured to contact the user's body, crotch area and legs.

11. The adult diaper device of claim 10, wherein the exterior surface is designed to contact clothing worn by the user.

12. The adult diaper device of claim 11, wherein the exterior surface comprises non-absorbent material and is impermeable to liquids.

13. The adult diaper device of claim 12, wherein the interior surface of the body component comprises absorbent materials.

14. An adult diaper device that maintains comfort and prevents leaks and accidents, the adult diaper device comprising:
a body component that comprises a central portion with an upper region that extends up to cover a user's waist and a lower region that covers a user's crotch area;
a pair of leg portions that have a top end and a bottom end, with the top end attached to the central portion and the bottom end defines leg openings that terminate on a thigh of a user's leg; and
absorbent material;
wherein the body component is configured as a pair of underwear with the pair of leg portions attached, which extend down a user's legs during wear;
wherein the lower region of the central portion defines a pair of upper thigh openings and the pair of leg portions downwardly depend from the pair of upper thigh openings;
wherein the body component comprises an elastic waistband that conforms to a user's waist;
wherein both sides of the body component comprise a tear-away seam;
wherein the absorbent material extends in a user's crotch area, up towards a user's back, and down the pair of leg portions to prevent leaks and maximize coverage;
wherein the body component comprises an exterior surface and an interior surface;
wherein the interior surface is dimensioned and configured to contact the user's body, crotch area and legs; and
further wherein the exterior surface is designed to contact clothing worn by the user.

15. The adult diaper device of claim 14, wherein the exterior surface comprises non-absorbent material and is impermeable to liquids.

16. The adult diaper device of claim 14, wherein the interior surface of the body component comprises absorbent materials.

17. The adult diaper device of claim 14, wherein the adult diaper device is a single-use article that may be discarded after one absorbent use.

18. The adult diaper device of claim 14, wherein the adult diaper device is manufactured in multiple sizes and shapes for a wide assortment of applications.

19. The adult diaper device of claim 14 further comprising a plurality of indicia.

20. A method of preventing embarrassing accidents and leaks when suffering with incontinence issues and IBS, the methods comprising the following steps:
providing an adult diaper device comprising a body component with extended leg portions;
inserting a user's legs into the extended leg portions and pulling up the device;
wearing the device throughout the day;
absorbing any leaks or accidents; and
utilizing the tear-away seam to remove the device after leaks or accidents for disposing in the trash.
